(19) United States
(12) Patent Application Publication
(10) Pub. No.: US 2018/0223239 A1
(43) Pub. Date: Aug. 9, 2018

(54) BIOFILM GROWTH DEVICES AND METHODS

(71) Applicant: Joshua D. Erickson, Champlin, MN (US)

(72) Inventors: Joshua D. Erickson, Champlin, MN (US); Christine E. Salomon, Saint Paul, MN (US)

(21) Appl. No.: 15/749,321

(22) PCT Filed: Aug. 9, 2016
(86) PCT No.: PCT/US2016/046110

§ 371 Date: Jan. 31, 2018

Related U.S. Application Data
(60) Provisional application No. 62/204,252, filed on Aug. 12, 2015.

(51) Int. Cl. C12M 1/12; C12M 3/06; C12M 1/00; B01L 3/00

(52) U.S. Cl. CPC C12M 25/02; C12M 23/16; C12M 29/10; B01L 2300/0848; B01L 2300/047; B01L 2300/0864; B01L 2300/0832; B01L 3/5027

(57) ABSTRACT

Embodiments include a biological growth device, and related methods for growing biofilms using embodiments of such biological growth device. The device includes a channel configured to convey a fluid medium. A first coupon well is disposed along the channel and defines an entry point for at least a portion of the fluid medium. A first coupon is configured to interface with the entry point defined by the first coupon well so as to receive the fluid medium. A second coupon well is disposed downstream along the channel from the first coupon well and defines an entry point for the fluid medium. A second coupon is configured to interface with the entry point defined by the second coupon well so as to receive the fluid medium subsequent to the first coupon.

### RELATED APPLICATIONS

This application claims priority to International Patent Application No. PCT/US2016/046110, filed on Aug. 9, 2016, which claims priority to U.S. Provisional Patent Application No. 62/204,252, filed on Aug. 12, 2015. The entire contents of both related applications are incorporated herein by reference.

### TECHNICAL FIELD

This disclosure relates generally to devices and methods for biofilm growth.

### BACKGROUND

Biofilms generally are relatively complex communities of microbial cells that are attached to a surface. Moreover, biofilms can represent a morphological state of many pathogenic microbes that can significantly augment their resistance to antimicrobial agents. The microbial cells encase themselves in a self-organized extracellular polymeric substance (EPS) that is, for example, primarily composed of proteins, polysaccharides, and extracellular DNA (eDNA). When these organisms attach to a surface and encase themselves in a substance (e.g., a biofilm) they are better protected from immune response and external stresses, such as antibiotics, chemicals and/or physical challenges. In their biofilm state, microbes can be, for example, 10 to 1000 times more resistant to antimicrobial treatment than planktonic cells.

One primary aspect of biofilm testing is reproducible growth of the biofilm in vitro. Conditions used to grow the biofilm can have a significant impact on the architecture of the biofilm itself as well as on performance of antimicrobial therapies. Particularly, fluid dynamics of the growth system and a surface on which the biofilm is grown can impact biofilm growth and/or resistance to antimicrobials. In order to create dynamic flow conditions substantially of a natural environment, an apparatus or reactor can be used to grow the biofilm.

### SUMMARY

This disclosure describes, in one aspect, a device designed to allow biological cells, such as, for example, prokaryotic and/or eukaryotic cells, to grow at an air-water interface on a surface of numerous coupons. Such coupons may be composed of a plurality of materials and under substantially continuous laminar-flow conditions. Specifically, this device is designed to allow bacteria and/or fungi to form biofilms on the surface of coupons at the air-water interface. Additionally, this device may be used for cell culture of, for instance, human cells, animal cells, plant cells, viruses, and/or protists. The design of the device can facilitate growth of said cells on a large number of coupons (e.g., 80-100) to enable, for instance, high-throughput testing to be performed. Additionally, the channels of said device can be designed so as to force growth media evenly over the surface of the one or more coupons.

In one embodiment, this device is re-usable and/or is machined out of an auto-clavable material. In other embodiments, the device may be a single-use design. That is, the device may be manufactured using a mold apparatus to form single-use devices out of plastic materials. In addition, embodiments include a device that is a closed-batch system where fluid media does not flow into and out of the device. Embodiments of the closed-system device may, for instance, be placed on an oscillator or other apparatus to enable fluid to flow over the surface of the coupons.

In another aspect this disclosure includes a generally circular, single-use version of the device that has one or more channels at or near a perimeter of the device that can include wells for one or more (e.g., multiple) coupons. This generally circular design may be designed for a single-use, but in other embodiments can be designed to be re-usable.

Embodiments further include methods for growing biofilms, such as by using any of the devices described herein. As one example, an embodiment of a method of growing a biofilm can include introducing a fluid medium into a first channel, arranging a first coupon to interface with an entry point defined by a first coupon well disposed along the first channel, and directing a portion of the fluid medium through the first channel and over a surface of the first coupon interfacing with the entry point defined by the first coupon well.

The above summary of the present invention is not intended to describe each disclosed embodiment or every implementation of the present invention. The description that follows more particularly illustrates exemplary embodiments. In several places throughout the description, guidance is provided through lists of examples, which examples can be used in various combinations (e.g., in addition to one another, or as alternatives). In each instance, the recited lists of examples serve only as representative groups and should not be interpreted as exclusive lists.

### BRIEF DESCRIPTION OF THE FIGURES

FIG. 1 is a schematic, perspective view of a drip-flow biofilm reactor.

FIG. 2 is a perspective view of an embodiment of a system setup including an embodiment of a MultiRep reactor.

FIG. 3A is a top plan view of the MultiRep reactor, with a top cover removed.

FIG. 3B is a perspective view of the MultiRep reactor of FIG. 3A including the top cover.

FIG. 3C is a cross-sectional view of a portion of FIG. 3A showing a configuration for promoting uniform biofilm growth over the surface of a coupon.

FIG. 4A is a depiction of a disc with no biofilm growth and having negative control.

FIG. 4B is a depiction of the disc of FIG. 4A with P. aeruginosa biofilm grown on the disc for a period of time in a microtiter plate and stained with crystal violet.

FIG. 4C is a depiction of the disc of FIG. 4A with P. aeruginosa biofilm grown on the disc, for the same period of time as the biofilm of FIG. 4B, but in the MultiRep reactor and also stained with crystal violet.

FIG. 5 is a chart showing a quantitative comparison of the P. aeruginosa grown in the microtiter plate of FIG. 4B with the P. aeruginosa grown in the MultiRep reactor of FIG. 4C.

FIG. 6A is a chart showing quantification of biofilm mass grown in the MultiRep reactor across various numbers of channels plotted against results from a crystal violet assay.

FIG. 6B is a chart showing quantification of biofilm mass grown in the MultiRep reactor across various numbers of channels plotted against results from an XTT/CFU enumeration assays.

FIG. 7 is a schematic, top plan view of an embodiment of a closed-batch plate design.

FIG. 8A is a schematic perspective view of a bottom of an embodiment of a generally circular closed-batch reactor.

FIG. 8B is a schematic, top plan view of the reactor of FIG. 8A.

### DETAILED DESCRIPTION OF ILLUSTRATIVE EMBODIMENTS

This disclosure, in general, describes a reactor for biofilm growth. In various embodiments, the biofilm reactor is a high-throughput laminar flow reactor that may be capable of producing uniform biofilm and/or cell culture growth on one or more surfaces. This can subsequently allow for efficient testing of the biofilm and/or cell culture growth.

The American Society for Testing and Materials International (ASTM) has outlined standard methods for use of, in particular, a drip flow reactor for biofilm growth (ASTM E2647-08). This drip flow reactor has been recommended to model multiple disease states such as, for example, chronic wound infections, lung infections, and urological infections. However, this current drip flow reactor is only capable of low throughput testing (4 coupons per growth cycle), and furthermore the biofilm growth on each coupon is generally not uniform.

However, in general standard methods that support biofilm claims are limited. The American society for Testing and Materials (ASTM) released a series of biofilm test methods in 2002-2011 (ASTM E2647-08, ASTM E2562-12, ASTM E2799-12, ASTM E2196-07). Each of the ASTM methods was designed for biofilm growth under different conditions. Table 1 below shows ASTM Standard methods for biofilm growth.

| | | Year | |
|---|---|---|---|
| Method | Reactor used | published | Growth conditions |
| ASTM E2196-07 | Rotating Disk | 2002 | Submerged, Continuous |
| | Reactor | | flow, Medium-shear |
| ASTM E2562-12 | CDC Reactor | 2007 | Submerged, Continuous |
| | | | flow, High-shear |
| ASTM E2647-08 | Drip Flow | 2008 | Air/liquid interface, |
| | Reactor | | Continuous flow, |
| | | | Laminar flow/low-shear |
| ASTM E2196-07 | MBEC | 2011 | Batch culture, low to |
| | | | medium-shear |

These ASTM methods are useful for standard testing, but generally do not support product efficacy claims with a governmental agency such as the EPA. The EPA, however, released the first SOPs for biofilm testing in August of 2013 that will support biofilm efficacy claims using the CDC reactor for biofilm growth (EPA MB-19-02, EPA MB-20-01). Recognizing that standardized biofilm testing is in the early stages of development, there is room for improvement of current methods and development of new biofilm growth methods that represent additional environments.

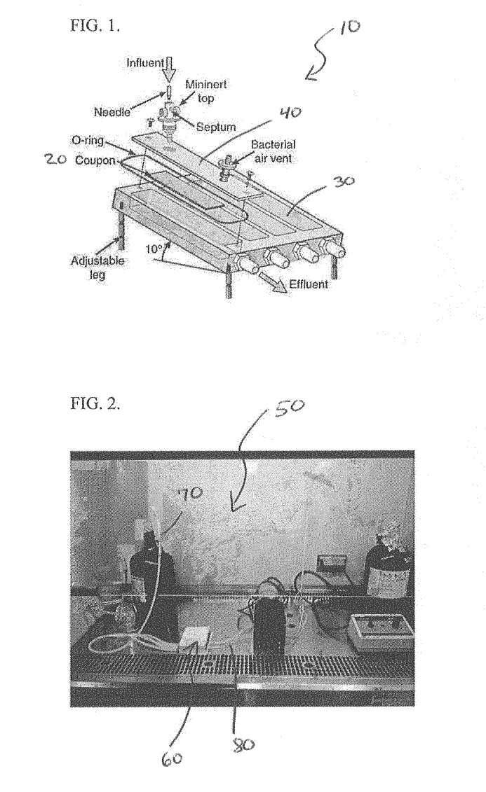

In order to create biofilm growth conditions that represent the environment in which biofilms grow near the interface of air and water under laminar-flow condition, a specific reactor is needed. Currently, a biofilm reactor that has been used in an attempt to fit these specifications is the drip flow reactor (ASTM E2647-08). FIG. 1 illustrates a schematic, perspective view of a general drip-flow biofilm reactor 10. As shown, the drip-flow biofilm reactor 10 accommodates four coupons 20 per growth cycle. Each of the four coupons 20 is disposed in corresponding recesses 30, and a cover 40 is secured in place over each recess 30. An influent is introduced through the cover 40 and dripped directly onto each coupon 20.

This drip-flow reactor 10, however, is inadequate for many reasons. First, the number of individual biofilm test replicates that can be produced from a single cycle of growth in this reactor is very low (e.g., 4 coupons/growth cycle as shown in FIG. 1). Second, biofilm growth resulting on each coupon is generally not uniform, which can result in statistically insignificant comparisons of antimicrobial products. Additionally, the time required to grow the biofilm is high for the number of replicates that are produced—the total time requirement is approximately 13 hours of active work distributed over a period of 5 days, yielding 195 minutes of active work per coupon.

Various embodiments of this disclosure provide for a laminar-flow biofilm growth reactor device (designated for exemplary purposes herein as the “MultiRep reactor”), and related methods, that yield substantially uniform, high-throughput biofilm growth. This laminar-flow biofilm reactor can be advantageous, for instance, in the design of new anti-biofilm treatments and in facilitating high-throughput testing of the substantially uniform biofilm growth.

FIG. 2 shows a perspective view of an embodiment of a biofilm growth system setup 50 including an embodiment of a MultiRep reactor 60. One or more influent lines 70 are in fluid communication with the MultiRep reactor 60 on one side, while one or more effluent lines 80 are in fluid communication with the MultiRep reactor 60 on another (e.g., opposite) side. As such, the biofilm growth system 50 is able to provide substantially continuous flow through the MultiRep reactor 60 in the illustrated embodiment.

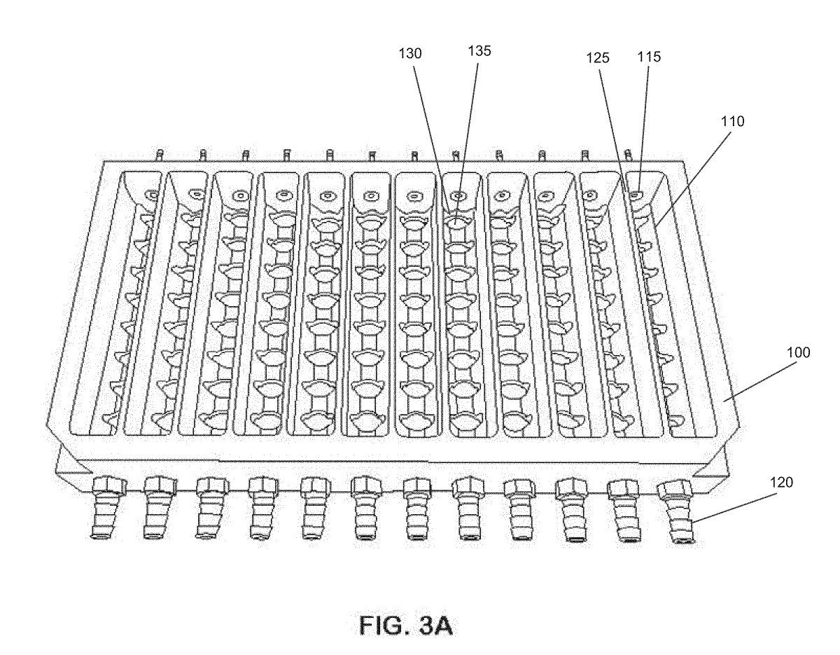

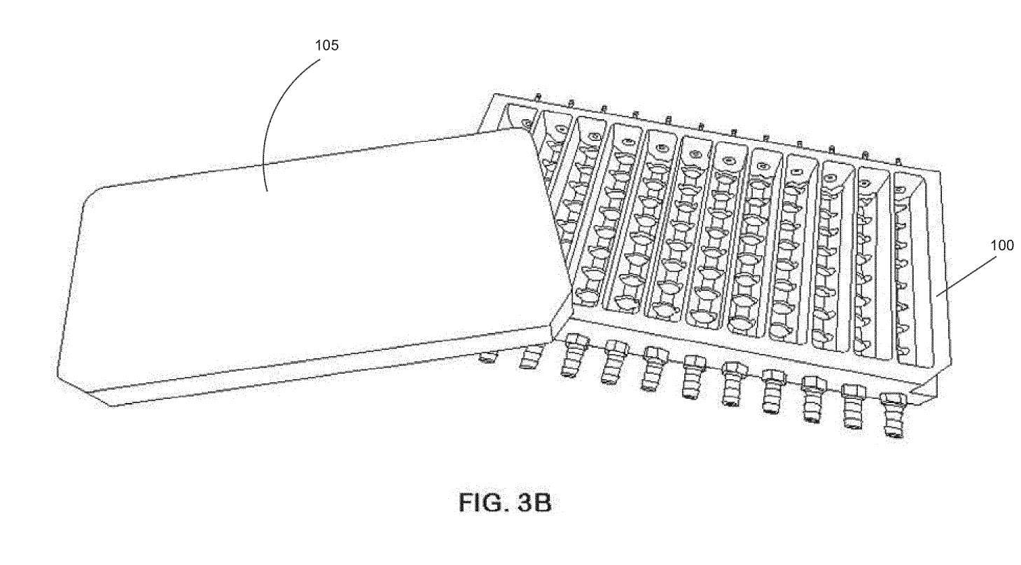

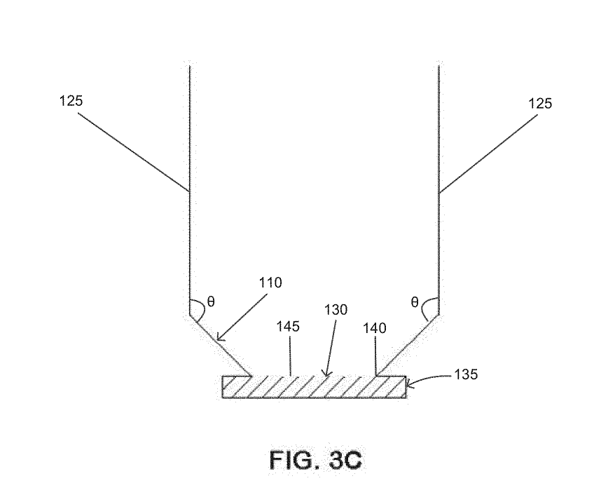

FIGS. 3A and 3B illustrate an embodiment of a MultiRep reactor 100, where FIG. 3A shows a top plan view of the MultiRep reactor 100 without a top cover and FIG. 3B shows a perspective view of the MultiRep reactor 100 of FIG. 3A including the top cover 105. The illustrated MultiRep reactor 100 may be used, for instance, in a system similar to that shown in FIG. 2. The MultiRep reactor 100 can provide a laminar flow biofilm growth device that produces uniform biofilms at an air-water interface and yields high replicates per growth cycle. In one example, the MultiRep reactor 100 may be CNC machined out of an autoclavable medical grade plastic. Depending on the desired application, the material can be selected such that the material is not compromised after repeated autoclaving procedures. Further, sterility testing has shown that such MultiRep reactors may be sterile after autoclaving, for instance, for approximately 15 min at 121° C. using a dry cycle.

The particular embodiment of the MultiRep reactor 100 shown in FIGS. 3A and 3B includes ten channels 110, with each channel 110 oriented in a direction extending from a respective inlet port 115 on a first end to a respective outlet port 120 on a second, opposite end. As shown, the channels 110 are arranged parallel to one another. In other embodiments, the MultiRep reactor 100 can include various other numbers of channels 110, depending, for example, on the desired biofilm yield per growth cycle, and these channels may be arranged in various orientations. Each inlet port 115 can be in fluid communication with an influent line, while each outlet port 120 can be in fluid communication with the effluent line so as to facilitate continuous fluid flow of a fluid medium through each channel 110 (see FIG. 2). For instance, at a flow rate of 0.70 mL/min into each channel the Reynolds number may be 29, which constitutes laminar flow. Partitions 125 may be included as shown to separate adjacent channels 110 from one another, such as to prevent fluid communication from one channel to another.

Each channel 110 in the example shown includes eight coupon wells 130. Each coupon well 130 may be associated with a distinct coupon 135 and define an entry point for communicating the continuous laminar flow of the fluid medium through the channel to a surface of the associated coupon 135. In particular, the surface of the coupon 135 that receives the fluid medium, through the entry point defined by the corresponding coupon well 130, may be configured at an interface between ambient air and the received fluid medium (e.g., an air-water interface, as opposed to fully submerged). The coupon 135 associated with each coupon well 130 can be arranged so as to interface (e.g., be in contact) with the entry point defined by the respective coupon well 135. In one example, a surface of the coupon 135 associated with each coupon well 130 can be arranged as close as possible to the coupon well 130, and thus the defined entry point, so as to provide a nearly flush flow path for the fluid medium communicated through the channel 110 as it passes over each of the coupon wells 130, defined entry points, and associated coupon 135 surfaces along the channel 110 (e.g. in a direction from the inlet port 115 toward the outlet port 120). In some embodiments, the coupon wells 130 can be substantially evenly spaced from one another along each channel 110.

In the described embodiment, each of the coupon wells 130 of the MultiRep reactor 100 can be designed to accommodate a coupon 135 that is approximately 5 mm in diameter. This coupon size can be optimal for transfer of discs, including the coupons, into a plate (e.g., a 96-well plate) for subsequent testing. The embodiment shown in FIGS. 3A and 3B, having ten channels 110 with eight coupon wells 130 and coupons 135 per channel, has capacity to grow eighty biofilms on separate coupons 135 during a single run. Thus, the described MultiRep reactor 100 has the ability to produce significantly greater biofilm yield per growth cycle as compared to currently used drip-flow reactors.

In operation, a fluid medium can be introduced into one or more (e.g., all) of the channels 110 of the MultiRep reactor 100, such as through the inlet port 115 corresponding to that particular channel. In other examples, there need not be a corresponding inlet port 115 for each individual channel 110, and rather a single inlet port can be used to communicate the fluid medium to all of the channels 110. The fluid medium may be introduced into one or more of the channels 110 as a continuous flow, and more particularly as a continuous laminar flow. The fluid medium can be directed along a channel 110 so as to encounter an upstream coupon well 130. The fluid medium can flow over the upstream coupon well 130 and, at a location where the upstream coupon well begins to define the entry point, the fluid medium can contact and flow over a surface of the coupon 135 corresponding to the upstream coupon well 130. The channel 110, coupon well 130, and coupon 135 surface may be configured so as to maintain laminar flow of the fluid medium within the channel 110. After passing over the surface of the coupon 135 associated with the upstream coupon well 130, the fluid medium can progress further downstream within the channel 110 to encounter a downstream coupon well 130 (e.g. a next adjacent coupon well 130 in the direction of flow of the fluid medium toward the outlet port 120). The fluid medium can flow over the downstream coupon well 130 and, at a location where the downstream coupon well 130 begins to define the entry point, the fluid medium can contact and flow over a surface of a coupon 135 corresponding to the downstream coupon well 130. The fluid medium can continue to be directed through the channel 110 over a number of coupon wells 130 and associated coupons 135. In the illustrated embodiment, the fluid medium is directed over eight coupon wells 130, eight coupon well entry points, and eight coupon 135 surface. In some embodiments of the MultiRep reactor 100, the fluid medium can be removed from the channel at the outlet port 120 on an end of the channel 110 opposite the inlet port 115. In further embodiments, the fluid medium may be directed from the outlet port 120 back to the inlet port 115 so as to be recycled through a channel again.

FIG. 3C shows a cross-sectional view taken along line A-A of FIG. 3A. The example configuration shown in FIG. 3C can promote uniform biofilm growth over a surface 145 of one or more coupons 135 associated with one or more coupon wells 130 along a channel 110 of the MultiRep reactor. As described previously and shown here, the surface 145 of the coupon 135 on which the biofilm grows can be arranged so as to interface (e.g., contact) the entry point 140 defined by the coupon well 130. In the example shown, the channel 110 can be configured to force fluid flow over the coupon well 130, entry point 140, and surface 145, and prevent flow of the fluid medium at regions of the channel 110 where the surface 145 will not receive the fluid medium. The channel 110 can intersect the partition 125 (e.g. on a fluid flow surface of the channel) at an angle θ that forces the flow of fluid medium away from the partition 125 and instead toward the surface 145. In some embodiments, the angle θ is greater than 90 degrees and less than 180 degrees. In other embodiments the angle θ is between 110 degrees and 160 degrees. In further embodiments, the angle θ is between 120 degrees and 150 degrees. In one embodiment, the angle θ is approximately 135 degrees. Each channel 110 of the MultiRep reactor can be configured similar to that described here and shown in FIG. 3C. Thus, the configuration of the MultiRep reactor can promote uniform biofilm growth over the surface of the coupons. As a result, an amount of biofilm on each coupon is maximized and variation between the coupons of the MultiRep reactor is minimized.

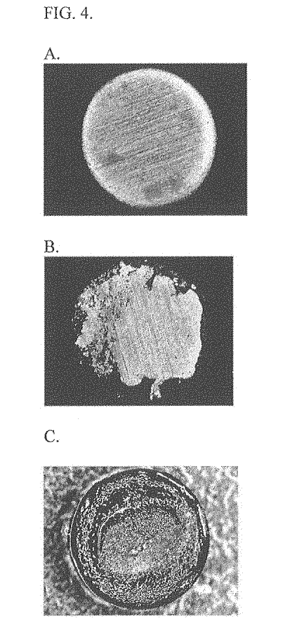

FIG. 4A illustrates a disc (e.g. stainless steel disc) without any biofilm growth, while FIGS. 4B and 4C illustrate P. aeruginosa biofilm grown on the disc of FIG. 4A. The biofilm shown in FIG. 4B was grown on the disc in a microtiter plate, as compared to the biofilm shown in FIG. 4C grown through use of a coupon in the described MultiRep reactor. The biofilm in both FIGS. 4B and 4C is illustrated as stained with crystal violet. As can be seen in comparing FIGS. 4B and 4C, a much more uniform biofilm over the surface of the coupon is achieved using the Multirep reactor (FIG. 4C). Whereas, the biofilm produced in the microtiter plate grew generally only around a periphery of the coupon (FIG. 4B).

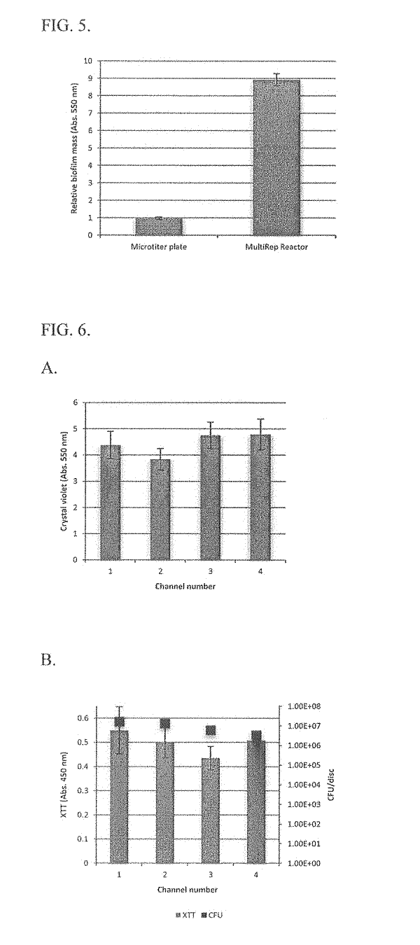

FIG. 5 shows a quantitative comparison of the amount of biofilm produced (by relative biofilm mass) when grown in the MultiRep reactor compared to a microtiter plate. While growth in the MultiRep reactor and growth on a microtiter plate are different (growth in the microtiter plate is a batch, submerged culture, while growth in the MultiRep reactor is at the air-water interface with continuous flow conditions), this comparison indicates that the MultiRep reactor is an effective tool for uniform, robust growth of biofilms at the air-water interface.

To determine if the coupon position has an effect on biofilm growth, studies have been performed that compared two measurements of biofilm growth. The first was a measurement of biofilm mass that was produced on each disc using the crystal violet assay. In this test, the crystal violet stain absorbs into the biofilm matrix and cells, and is dissolved/extracted with an acid. The resulting absorbance represents the amount of biofilm that was initially present. The second measurement of biofilm growth was cell viability. For this measurement, both the XTT assay and CFU enumeration were performed. The XTT assay is a colorimetric test that detects metabolically active cells. Due to the peristaltic pump that was used for this study, only four channels were tested. This generated a sample population of 32 coupons (4 channels×8 coupons/channel). The MultiRep reactor was capable of generating biofilms on each of the 32 coupons that were tested. The biofilm growth across the four channels and down each row were analyzed and compared.

The average values obtained for each channel (e.g., channel number 1, channel number 2, etc.) are shown in FIGS. 6A and 6B. The results for the crystal violet assay are shown in FIG. 6A, and the results for the XTT assay and CFU enumeration are shown in FIG. 6B. The data generated from these assays were statistically analyzed with an ANOVA test. The crystal violet, XTT, and CFU enumeration values statistically correlated and indicate that there was no substantial difference between biofilm mass or cell viability across the channels. The crystal violet assay data resulted in a p-value of 0.52, and the XTT assay resulted in a p-value of 0.65. Therefore, the coupon position across each channel has no statistically significant impact on biofilm growth.

The MultiRep reactor may be designed to accommodate coupons that are a proper size for transfer to a particular plate as desired for a specific application. For example, in one application the MultiRep reactor can be configured to accommodate coupons sized for transfer to a 96-well microtiter plate, for use in testing and cell recovery, for instance. To demonstrate this utility, biofilms grown on steel discs in the MultiRep reactor were transferred to a 96-well plate, treated with anti-microbial products, and compared using the XTT assay. From the total coupons used in this study, 4 (1 from each channel) were CFU enumerated, 4 (1 from each channel) were measured using the crystal violet assay, and 16 were used to compare the impact that multiple treatments had on cell viability. The CFU enumeration and crystal violet assays were performed to compare the starting CFU and biofilm mass from coupons across different channels. Results showed that, following biofilm growth in the MultiRep reactor, coupons can be transferred to a 96-well plate, treated with antimicrobials and analyzed to differentiate the antimicrobial effect on the biofilm. This demonstrates the feasibility of the MultiRep reactor for antimicrobial product development against microbial biofilms.

In an exemplary application of the MultiRep reactor, we evaluated the growth of C. albicans biofilms to characterize anti-fungal natural products that have synergist activity with copper against this pathogen. Briefly, a Streptomyces sp. bacterium (CES-254) was isolated from the Soudan mine in northern Minnesota, and was found to have anti-fungal activity. Further testing indicated that this organism produced a suite of compounds that are synergistic with copper against C. albicans planktonic cells. Preliminary testing against C. albicans biofilms grown on steel discs in a 96-well plate indicated that this synergy might also be effective against this organism in the biofilm state. The MultiRep reactor may be an important tool for further characterization of this activity and the discovery of alternative components useful for anti-fungal therapies.

With a growing understanding of the importance of biofilms in antimicrobial efficacy testing, there is a need for reactors that support biofilm growth under a wide range of conditions. Current reactor options are limited to low replication and less than ideal biofilm uniformity over the surface of the coupon. This results in a high cost per replicate along with a high time requirement per replicate. The presently described MultiRep biofilm reactor can produce biofilm growth at the air-water interface under laminar flow conditions. The MultiRep reactor can yield a high number of replicates per growth cycle with uniform biofilm growth over the surface of the coupon. High replication per growth cycle drastically lowers the cost per replicate and the time required per replicate. The coupon position within the MultiRep reactor was found to have no statistically significant impact on biofilm growth across each channel and the ease of subsequent testing in a 96-well plate was demonstrated. The MultiRep reactor may therefore be used in a method for biofilm growth under laminar flow conditions at the air-water interface.

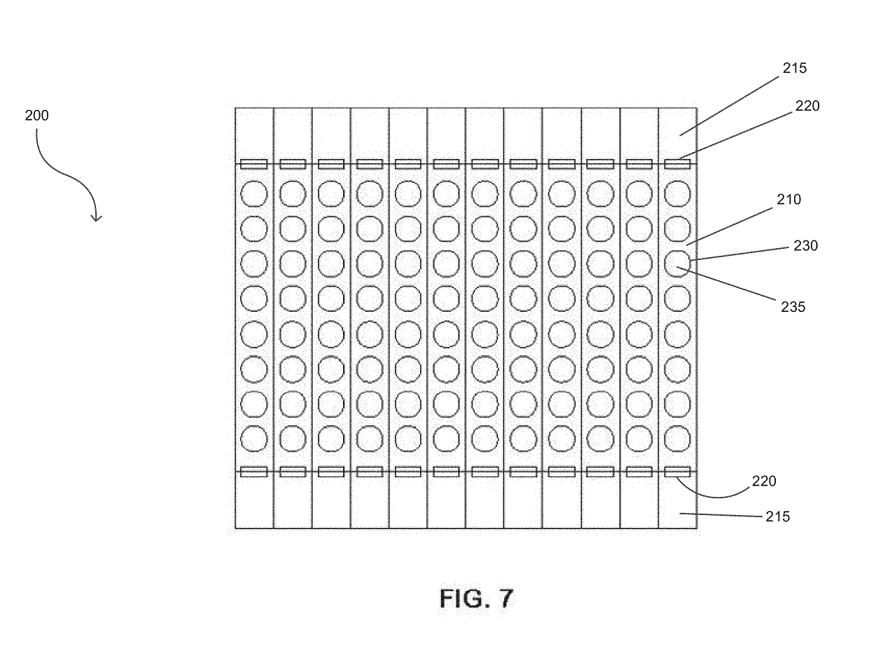

Other embodiments of the MultiRep reactor can be constructed to achieve similar benefits. One example includes a closed-batch device. One example of a closed-batch device is illustrated in FIG. 7, which shows a schematic top plan view of the embodiment of the closed-batch reactor 200. The reactor 200 can be similar to the MultiRep reactor described previously, except that the reactor 200 is a closed system where continuous supply of a fluid medium external to the reactor 200 is not provided. Instead a fluid medium can be placed into a media basin 215 and communicated into and through each channel 210 via a basin port 220, rather than continuously supplied via externally communicated fluid lines. As such, the fluid media may not leave the reactor 200 during operation.

In one application, the reactor 200 can be a single-use reactor. As shown, the exemplary reactor 200 includes twelve channels 210, with each channel 210 having eight coupon wells 230, where distinct coupons 235 can be arranged to interface with each coupon well 230. To facilitate flow of the fluid media from a media basin 215, into a channel 210, and over a surface of one or more coupons 235, the reactor 200 can be placed on an oscillator. For instance, the oscillator can be in one application a tilting oscillator which can direct the fluid media from the media basin 215, through an extent of the channel 210 (and thus over all coupon surfaces associated with all coupon wells of the particular channel), and into a media basin 215 on an opposite longitudinal end of the channel 210. Other channels 210 of the reactor 200 can have fluid media communicated therethrough in a similar manner.

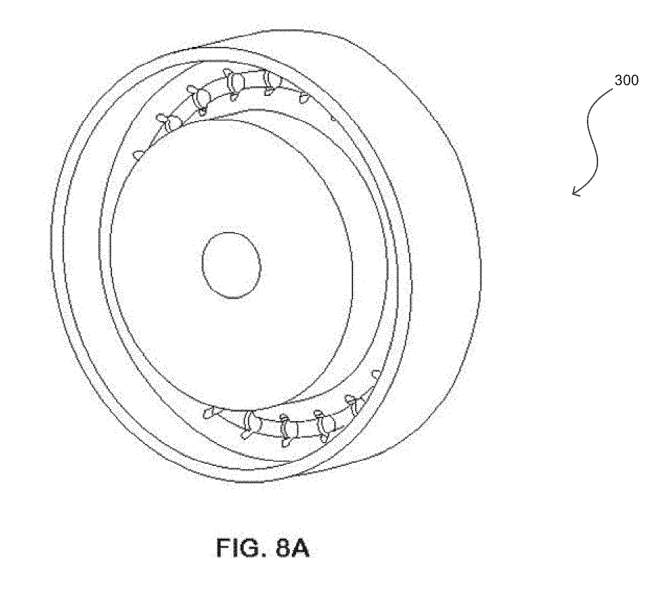

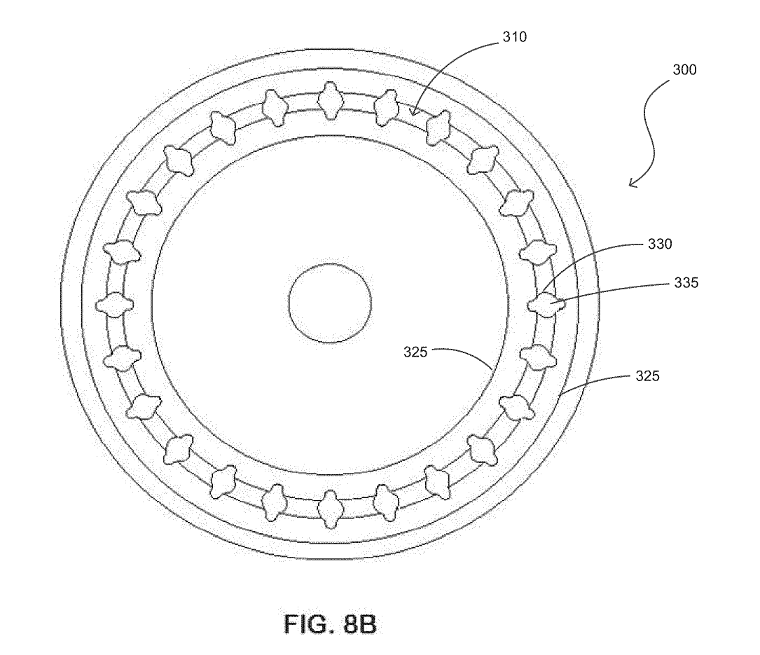

FIGS. 8A-8B illustrate another embodiment of a closed-batch reactor 300. FIG. 8A shows a schematic perspective view of a bottom of the closed-batch reactor 300, while FIG. 8B shows a top plan view of the closed-batch reactor 300. The reactor 300 can be similar to the MultiRep reactor described previously, except that the reactor 300 is a closed system where continuous supply of a fluid medium external to the reactor 300 is not provided. Instead a fluid medium can be placed (e.g., manually) into a channel 310 of the reactor 300. The reactor 300 defines a generally circular geometry, with the channel 310 located at or near a perimeter of the reactor 300. For instance, as shown the channel 310 is bounded by partitions 325 on each side of the channel 310, where one partition is located substantially at the perimeter of the rector 300. Coupon wells 330 and associated coupons 335 can be spaced along the channel 310, and in the illustrated example the reactor 300 includes twenty-one coupon wells and associated coupons.

Given that the reactor 300 is a closed-batch reactor, various means can be employed to circulate the fluid media along the channel 310 and over the respective growth surfaces of the coupons 335. In one instance, the reactor 300 can be placed on an orbital shaker so as to cause the fluid media introduced into the channel 310 to flow along the channel 310 and over each of the coupon wells 330 and associated coupons 335. By locating the channel 310 at or near a perimeter of the generally circular reactor 300, the fluid media can be circulated along the channel 310 more easily by the orbital shaker than in examples where the channel 310 is located closer to a center point of the reactor 300. In some examples, the reactor 300 can be configured to facilitate a stacked arrangement of multiple reactors 300, for example on the orbital shaker. By stacking multiple reactors one on top of the other, a greater number of coupons can be utilized, and thus a greater number of uniform biofilm growth samples can be obtained. In some applications, the one or more reactors 300 can be designed as single-use reactors.

Embodiments further include methods for growing biofilms, such as by using any of the devices described herein. As one example, an embodiment of a method of growing a biofilm can include introducing a fluid medium into a first channel, arranging a first coupon to interface with an entry point defined by a first coupon well disposed along the first channel, and directing a portion of the fluid medium through the first channel and over a surface of the first coupon interfacing with the entry point defined by the first coupon well.

In further embodiments, the method can include arranging a second coupon to interface with an entry point defined by a second coupon well disposed along the first channel, and directing the portion of the fluid medium through the first channel and over a surface of the second coupon interfacing with the entry point defined by the second coupon well. In some examples, the portion of the fluid medium is directed over the surface of the first coupon and the surface of the second coupons at different times. As noted previously, in many instances the portion of the fluid medium is directed over the surface of the first coupon and the surface of the second coupon as laminar flow.

Embodiments of the method can further include introducing the fluid medium into a second channel at time when the fluid medium is introduced into the first channel, arranging a third coupon to interface with an entry point defined by a third coupon well disposed along the second channel, and directing a portion of the fluid medium through the second channel and over a surface of the third coupon interfacing with the entry point defined by the third coupon well.

As used herein, the term “and/or” means one or all of the listed elements or a combination of any two or more of the listed elements; the terms “comprises” and variations thereof do not have a limiting meaning where these terms appear in the description and claims; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

In the preceding description, particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

Strains and Growth Conditions

For routine growth, Pseudomonas aeruginosa ATCC 15442 was taken from a frozen glycerol stock (−80° C.) and plated on TSA. Single colonies were used to inoculate trypticase soy broth (TSB) (30 g/L), and cultures were grown at 37° C. for 18-24 h in a shaker (200 rpm). For growth in the MultiRep reactor, TSB media was made at a concentration of 6 g/L and the temperature was 23° C.

MultiRep Biofilm Reactor Method

Reactor Preparation:

Autoclaved stainless steel coupons (5 mm diameter) were rinsed twice with de-ionized water and placed into the wells of the MultiRep reactor with a forceps. The tubing was then assembled and attached to the reactor vessel and autoclaved. Silicone tubing was used for the effluent port attachments (VWR ⅛″×¼″ Cat. #89068-432) and the influent port attachments (Masterflex L/S 14 tubing Cat. #96400-14). This is the correct size tubing for the inlet and outlet adaptors of the reactor, and also enabled the low flow rate that was desired for the system. A glass flow break was added to the system upstream from the peristaltic pump. Glass flasks (4 L) were used for nutrient supply and waste. TSB medium (6 g/L) was autoclaved in 2 L volumes and added to the sterilized glass flask used for nutrient supply. The waste flask was attached to a vacuum line in order to efficiently pull the waste media from the reactor (see FIG. 2 for image of reactor system). The system was set up inside of a biological safety hood with controlled airflow to minimize contamination.

Reactor Inoculation:

A 5 mL culture of P. aeruginosa (ATCC 15442) was inoculated with an isolated colony from trypticase soy agar (TSA). The 5 mL culture was incubated overnight at 37° C. and 200 rpm for 18-24 h, and then diluted 1:10 into fresh TSB media. The tubing on both the inlet and outlet ports of the reactor was clamped off and 4 mL of the diluted culture was added to each test channel in the reactor. The inoculated system was incubated at 23° C. for 4 hours to allow the cells to adhere to the surface.

Continuous Flow Phase:

The clamps were then removed from the tubing and the reactor was set to an angle by adding a 5 mm spacer underneath the inlet side of the reactor. The pump used in this study was a MasterFlex Pump 3 (Model #7553-71) with an Easy Load II pump head (Model #77202-60). The pump speed was set at level 1, which resulted in a flow rate of ˜0.7 mL/min. The continuous flow system was then run for 24 hrs. If the biofilm needed to be grown for a longer period of time (48-72 hrs.), the waste was removed and sterile media was added to the feed flask every 24 hrs.

Crystal Violet Assay:

This method was adapted from a previous method (O'Toole, 2011). Briefly, discs were transferred to a round bottom 96-well plate and washed 1× with 160 μL of sterile PBS (pH 7.2) using a multichannel pipette. 150 μL of crystal violet (0.1%) was then added to each well. Discs were soaked in crystal violet for 10-15 min, and washed 3× with 160 μL of PBS. The discs were then transferred to clean wells and washed 1 final time with 160 μL of PBS. 160 μL of glacial acetic acid (30%) was then added to each of the wells and incubated at room temperature for 10-15 min. Following this incubation period, the acetic acid solution was pipetted up and down 2 times and transferred to clean wells of a 96 well flat-bottom plate. Absorbance was read in a Bio-Tek plate reader at 550 nm.

XTT Assay:

Following treatment of the discs, discs were transferred to a round bottom 96-well plate and washed 1× with 160 μL of sterile PBS (pH 7.2). 2,3-Bis-(2-Methoxy-4-Nitro-5-Sulfophenyl)-2H-Tetrazolium-5-Carboxanilide) sodium salt (XTT) was added to warm PBS (55° C.) at a concentration of 0.8 mg/mL. This solution was vortexed and centrifuged for 1 minute to pellet the insoluble material. Menadione was added to DMSO at a concentration of 0.2 mg/mL. 25 μL of the XTT solution, 1 μL of the menadione solution, and 74 μL PBS were added to each well of the plate. The plate was incubated in the dark for a minimum of 6 h at 37° C. Following the incubation period, the XTT solution was pipetted up and down twice and transferred to a new microtiter plate. The absorbance was then read at 450 nm using a Bio-Tek plate reader.

CFU Enumeration:

Following treatment of the discs, the discs were transferred to a round bottom 96-well plate and washed 1× with sterile PBS (pH 7.2). 150 μL of sterile PBS was added to each well that contained a disc. The plate was then sealed inside of a plastic bag, and placed in a water bath sonicator (sonicated on high for 30+/−5 min). A serial 10 fold dilution of each disc was then carried out in additional 96-well microtiter plates. After sonication, the content of each well was pipetted up and down 2 times. Then, 100 μL from each well containing a disc was transferred to the top row of a sterile flat-bottom 96-well microtiter plate. 180 μL of sterile PBS was added to each well in rows B-H of the plate. The transferred 100 μL samples were then serial diluted (10⁰-10⁻⁷) by transferring 20 μL from each well into the next using a multichannel pipette. Each well was mixed by pipetting 2 times and swirling the pipette tips in the well a total of ten revolutions. Fresh pipette tips were used for each subsequent transfer. The contents of each dilution were then spot plated on TSA using a multichannel pipette by first mixing each well and spotting 10 μL of the sample onto the TSA. Plates were incubated at 35° C.+/−2° C. for 16-18 h. This method was adapted from the MBEC ASTM method (ASTM E2799-12).

Calculation of CFU/Disc:

Log 10(CFU/disc)=Log 10[(A/B)(C)(D)]

Where:

A=CFU counted in the spot

B=Volume plated

C=Well volume

D=Dilution

Reynolds Number Calculation:

Calculation of the Reynolds number for the MultiRep reactor was based on an equation developed for fluid flow through an inclined plane channel (Bird et al, 2002). The calculations were based on the bulk fluid being water at 20° C. The fluid flow was determined to be 0.7 mL min⁻¹. The fluid thickness was determined to be 1.2 mm based on the flow rate and the geometry of the channel.

Statistical Analysis

The data generated from the crystal violet assay, XTT assay, and CFU enumeration was statistically analyzed using a one-way ANOVA test. The results were generated with 3 degrees of freedom between groups, and 28 degrees of freedom within groups for the comparison of the channels. For the comparison of the rows, the results were generated with 7 degrees of freedom between groups, and 28 degrees of freedom within groups.

The complete disclosure of all patents, patent applications, and publications, and electronically available material cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

What is claimed is:

1. A biological growth device comprising:
a first channel configured to convey a fluid medium;
a first coupon well disposed along the first channel and defining an entry point for at least a portion of the fluid medium, wherein a first coupon is configured to interface with the entry point defined by the first coupon well so as to receive the fluid medium; and
a second coupon well disposed downstream along the first channel from the first coupon well and defining an entry point for the fluid medium, wherein a second coupon is configured to interface with the entry point defined by the second coupon well so as to receive the fluid medium subsequent to the first coupon.

2. The device of claim 1, wherein the first channel is disposed at or near a perimeter of the device, and wherein the device defines a generally circular geometry.

3. The device of claim 1, wherein the first channel is part of a closed batch system.

4. The device of claim 3, further comprising:
an oscillator configured to convey the fluid medium through at least a portion of the first channel.

5. The device of claim 1, further comprising:
a second channel configured to convey a fluid medium;
a third coupon well disposed along the second channel and defining an entry point for at least a portion of the fluid medium, wherein a third coupon is configured to interface with the entry point defined by the third coupon well so as to receive the fluid medium; and
a fourth coupon well disposed downstream along the second channel from the third coupon well and defining an entry point for the fluid medium, wherein a fourth coupon is configured to interface with the entry point defined by the fourth coupon well so as to receive the fluid medium subsequent to the third coupon.

6. The device of claim 5, wherein a partition separates the first channel and the second channel, and wherein the first channel intersects the partition at an angle greater than 90 degrees and less than 180 degrees.

7. The device of claim 5, wherein the first channel is in fluid communication with a first inlet port on a first end of the first channel and with a first outlet port on a second end of the first channel, and wherein the first inlet port is configured to introduce the fluid medium into the first channel.

8. The device of claim 7, wherein the second channel is in fluid communication with a second inlet port on a first end of the second channel and with a second outlet port on a second end of the second channel, and wherein the second inlet port is configured to introduce the fluid medium into the second channel.

9. The device of claim 8, wherein the first channel extends from the first inlet port to the first outlet port and the second channel extends from the second inlet port to the second outlet port, and wherein the first channel extends parallel to the second channel.

10. A biological growth device comprising:
a first channel in fluid communication with a first inlet port on a first end of the first channel and a first outlet port on a second end of the first channel, wherein the first inlet port is configured to introduce a fluid medium into the first channel;
a first coupon well disposed along the first channel and defining an entry point for at least a portion of the fluid medium; and
a first coupon arranged to interface with the entry point defined by the first coupon well so as to receive the fluid medium.

11. The device of claim 10, further comprising:
a second coupon well disposed along the first channel and defining an entry point for the fluid medium; and
a second coupon arranged to interface with the entry point defined by the second coupon well so as to receive the fluid medium.

12. The device of claim 11, further comprising:
a second channel in fluid communication with a second inlet port on a first end of the second channel and a second outlet port on a second end of the channel, wherein the second inlet port is configured to introduce the fluid medium into the second channel;
a third coupon well disposed along the second channel and defining an entry point for at least a portion of the fluid medium; and
a third coupon arranged to interface with the entry point defined by the third coupon well so as to receive the fluid medium.

13. The device of claim 12, wherein the first channel extends from the first inlet port to the first outlet port and the second channel extends from the second inlet port to the second outlet port, and wherein the first channel extends parallel to the second channel.

14. The device of claim 12, further comprising:
a fourth coupon well disposed along the second channel and defining an entry point for the fluid medium; and
a fourth coupon arranged to interface with the entry point defined by the fourth coupon well so as to receive the fluid medium.

15. The device of claim 11, wherein the first channel is configured to provide laminar flow of the fluid medium.

16. A method of growing a biofilm, the method comprising the steps of:
introducing a fluid medium into a first channel;
arranging a first coupon to interface with an entry point defined by a first coupon well disposed along the first channel; and
directing a portion of the fluid medium through the first channel and over a surface of the first coupon interfacing with the entry point defined by the first coupon well.

17. The method of claim 16, further comprising:
arranging a second coupon to interface with an entry point defined by a second coupon well disposed along the first channel; and
directing the portion of the fluid medium through the first channel and over a surface of the second coupon interfacing with the entry point defined by the second coupon well.

18. The method of claim 17, wherein the portion of the fluid medium is directed over the surface of the first coupon and the surface of the second coupons at different times.

19. The method of claim 18, wherein the portion of the fluid medium is directed over the surface of the first coupon and the surface of the second coupon as laminar flow.

20. The method of claim 17, further comprising:
introducing the fluid medium into a second channel at time when the fluid medium is introduced into the first channel;
arranging a third coupon to interface with an entry point defined by a third coupon well disposed along the second channel; and
directing a portion of the fluid medium through the second channel and over a surface of the third coupon interfacing with the entry point defined by the third coupon well.
